(19) United States
(12) Patent Application Publication
(10) Pub. No.: US 2025/0057783 A1
(43) Pub. Date: Feb. 20, 2025

(54) ORAL FILMS OF ANTIPRURITIC DRUGS

(71) Applicant: Laurus Labs Limited, Hyderabad (IN)

(72) Inventors: Siva Ramakrishna VELAGA, Hyderabad (IN); Siva Ramakrishna RAYALA, Hyderabad (IN); Srikant PIMPLE, Hyderabad (IN); Ananda Haranath UPPALA, Hyderabad (IN)

(21) Appl. No.: 18/723,444

(22) PCT Filed: Dec. 29, 2022
(86) PCT No.: PCT/IN2022/051134

§ 371 Date: Jun. 23, 2024

(30) Foreign Application Priority Data
Dec. 30, 2021 (IN) 202141062002

(51) Int. Cl. A61K 9/70; A61K 9/00; A61K 31/519; A61K 38/13; A61K 47/02; A61K 47/10; A61K 47/26; A61K 47/36; A61K 47/46

(52) U.S. Cl. CPC A61K 9/7007; A61K 9/006; A61K 31/519; A61K 38/13; A61K 47/02; A61K 47/10; A61K 47/26; A61K 47/36; A61K 47/46

(57) ABSTRACT

The present invention relates to new oral dosage forms of Antipruritic drug(s) and its pharmaceutically acceptable salts thereof for veterinary use, such as for oral administration to animals. More specifically the present invention provides compositions and process for preparing oral film comprising Antipruritic drug(s) and its pharmaceutically acceptable salts thereof.

### FIELD OF THE INVENTION

The present invention relates to new oral dosage form of Antipruritic drugs and its pharmaceutically acceptable salts thereof, for veterinary use, such as for oral administration to animals. More specifically the present invention relates to oral film compositions comprising antipruritic drugs and its pharmaceutically acceptable salts thereof and method of administering the same to the oral cavity of the animals in treating, preventing or controlling pruritus caused due to atopic or allergic dermatitis.

### BACKGROUND OF THE INVENTION

Due to a rapid growth in per capita income globally and improvement of people's living standards, the culture of maintaining animals or domestic pets as house hold has raised. The pet population has grown drastically over the period of year. Dogs are the most popular pets in households, while cats have become the second-preferred option, followed by fish and birds. Other pets, such as dogs used for police work and search and rescue operations, race horses, etc., each play a significant role in society. On another side, Animal husbandry and Dairying is a critical industry for ecological and environmental survival. Animal husbandry requires continuous nurturing of animals for meat, fibre, milk or for aiding agricultural purposes. As the Animal husbandry, Dairying and pet culture diversified over years, effective veterinary care is also growing on par with culture. Currently, veterinary care has become very important to the animal owners as well as the rest of society. The proper and efficient veterinary care of these pets and other animals is very important for overall ecosystem development.

Accordingly, demand for high quality and ease of administering the veterinary medicines has also seen a growth, as these animals are been prone to many illness and injuries. The most regular clinical illnesses suffered by animals Dermatitis or Itchy inflamed skin disease caused by an over reactive or hypersensitive immune system triggered by an allergic reaction to airborne substances like pollens of Grass, Trees and Weeds, House dust, Household chemicals, Dust mites, Food Allergies, Flea Allergies, Lice, Ticks, Bacterial hypersensitivity and Worms. Besides, periodical medication with antibiotics, anti-inflammatory agents, anti-viral agents, vaccines and wormers, occasional medications like cancer chemotherapy could cause over reactive of immune system which may results in atopic or allergic dermatitis, that induce severe pruritus in animals.

Accordingly, Atopic dermatitis is an exaggerated IgE-mediated immune response often caused by genetic mutation or propensity of allergy and Allergic dermatitis is any exaggerated immune response to a foreign antigen. The primary clinical sign of Atopic or Allergic dermatitis is Pruritus i.e. severely Itchy and/or Inflammatory skin condition, animals exhibits the same by Scratching, Biting, Rubbing and Licking the effected part. The secondary clinical signs of dermatitis are erosions, ulcerations, hair loss, skin scaling, scales piercing hairs, tumours, odour, and/or greasy seborrhoea. The animals become lethargic due to severe pruritus.

To treat or prevent of such inducement or triggering of pruritus associated with allergic dermatitis and atopic dermatitis, there are several effective Antipruritic drugs approved by veterinary drug regulatory authorities globally.

It is known that the successful treatment associated with dermatitis depends on identifying the underlying cause. Following are the some of the well known approved Antipruritic drugs identified as antihistamines like Cetirizine, Diphenhydramine, Chlorphenamine, and Hydroxyzine; oral Glucocorticoids like Prednisolone, Prednisone, Methylprednisolone, Hydrocortisone, Betamethasone and Flumethasone; calcineurin inhibitor like Cyclosporine; Janus Kinase inhibitor (JAK-1) like Oclacitinib; fatty acids like Omega-3 and Omega-6; psychogenic drugs like Triflupromazine, Trimeprazine Tartrate or Alimemazine Tartrate, Loratadine, Fluoxetine, Amitriptyline, Norepinephrine, Clemastine or meclastin fumarate; opioid antagonists like Dextromethorphan and Naltrexone. All the above drugs are available as tablet, capsule, oral solution or injection dosage forms.

It is also know from the art that, Antihistamines acts by competitively antagonising histamine binding receptors (H1-receptors) there by it reduces dermal inflammation and prevent allergic reactions that cause pruritus. Glucocorticoids are also known to be the most effective drugs in the management of pruritus by modulating or compromising hyper immune response triggered in the effected animal. Janus Kinase inhibitors block expression of proinflammatory cytokines by inhibiting signal transmission JAK pathway. Fatty acid supplementation produces anti-inflammatory leukotriene and prostaglandins, which is believed to prevent pruritus by reducing dermal allergic or inflammatory reactions. Calcineurin inhibitors prevent the induction of genes encoding for cytokines (IL 2 and 4) and their receptors, thus affecting both humoral and cellular immune responses. Psychogenic drugs block reuptake of serotonin by nerves thereby increase serotonin levels in the brain and Opioid antagonists, which is believed to enhance the mood of the animals by reducing the urge to scratch, lick or bite caused out of pruritus.

Oclacitinib, a Janus Kinase inhibitor (JAK-1), as Oclacitinib maleate was approved in USA as film-coated tablets (3.6 mg, 5.4 mg and 16 mg) dosage form on May 14, 2013 to Zoetis (a Pfizer subsidiary) for Control of pruritus associated with allergic dermatitis and control of atopic dermatitis in Dogs under the brand name APOQUEL®.

Methylprednisolone acetate, a glucocorticoid, is approved in USA under the brand name Medrol® as 1 mg, 2 mg, or 4 mg oral tablet and Depo Medrol® as 20 mg/ml or 40 mg/ml injectable dosage forms for treating allergic and dermatologic disorders in Cats, Dogs and Horses.

Prednisolone, a glucocorticoid, as prednisolone acetate or prednisolone sodium succinate or prednisolone sodium phosphate were approved in USA under the brand name PrednisTab® as 5 mg to 25 mg oral tablets and Solu-Delta Cortef© as 20 mg/ml or 25 mg/ml injectable dosage form to administer in Dogs and Cats for treating allergic inflammatory dermatitis.

Cyclosporine, a Calcineurin inhibitor, was approved in USA under the brand name Atopica® 10 mg, 25 mg, 50 mg, or 100 mg per capsules, 100 mg/ml oral solutions to administer in Dogs and Cats to treat atopic and allergic dermatitis.

CA2965524 patent discloses and claims soft chew veterinary composition comprising: a. Oclacitinib or the maleate salt thereof, b. at least one binding agent; c. at least one disintegrate; d. at least one wetting agent; and e. at least one flavouring and its use as antipruritic in animals.

CN109172547 patent publication discloses self-micro emulsifying oral quick-dissolving films for animals, which comprises self-microemulsifying component, film-forming material, plasticizer, disintegrant, thickener, and flavouring agent. Self-micro emulsifying components are composed of an oil phase, an emulsifier and a co-emulsifier; the oil phase can be castor oil, olive oil, oleic acid, ethyl oleate, medium chain triglycerides, a mixture of one, two or more of glyceryl oleate and isopropyl myristate; the emulsifier can be selected from polyethylene glycol glycerides, polysorbates, and polyoxyethylene castor oils. The co-emulsifier can be one, two or more mixtures of ethanol, propylene glycol, glycerin, polyethylene glycol, and diethylene glycol monoethyl ether.

WO2020172232 patent publication discloses and refers a stable, palatable, soft chewable oral composition for increased acceptance of the medication in animals, which show resist swallowing hard tablets or capsules and chewing bitter and/or granular dosage forms. Specification discloses suitable non-limiting excipients of an animal based palatant, a non-animal based palatant, a flavour modifier, and at least one veterinary acceptable excipient that is selected from at least one each of a disintegrant, binder, lubricant, humectant, and glidant; and the soft chewable tablet is compressed with a rotary tablet press. Though a simple method of administering but it is limited by several additional factors such as complete and sufficient chewing of the dosage form, capacity of animal or pet to chew, reluctance and compliance of the animal or pet to chew, splitting out the half chew or dogs drooling etc., which effects the release of drug and effective treatment.

U.S. patent publication No. 20050136096 discloses edible films for administration of medicaments to animals, wherein the edible film disclosed has an applied coating and a thin film. The applied coating is a powder matrix including one or more medicaments optionally with a carrier is applied as a coating by Sifting, Screening, atomization, Static, mechanical agitation or fluidization etc. Though the disclosure does not disclose any antipruritic drugs or films comprising the same, such edible films are limited by animal's oral mucosa is being directly exposed to the drugs applied over the film, that may be cause sense of irritability or drooling when administered, resulting in dosing error and reluctance to take such films from next dose onwards. Also, the coated medicament on the thin film may be eroded during the process of administration i.e., while taking out from the package and handling it while administering to the animal.

It is well known from the arts that the methods of administering Antipruritic drugs and its pharmaceutically acceptable salts thereof to animals or pets include vascular injections, muscular injections and oral administration of liquids or tablets or pills or soft chewables or drug coatings on thin oral films. Disadvantages of these known pharmaceutical delivery methods include difficulty swallowing, difficulty in administration, inconvenience to pet medical staff and pet owners, pain to the animals or pets, cause damage to oral cavity or oesophagus with auxiliary feeders, erosion of drug coating and difficulty in measuring the dosages when liquids or tablets or pills are added to animal feed, since, for example, some time a number of animals or pets will share a common feed container and animals will sometimes try to spit out the medicament, which reduces therapeutic effect in the target animals.

Parenteral dosage forms are difficult in administration, also cause pain to the animals, maximum attention while administering and pose a danger of self-administering or injury to the administrating volunteer like pet nursing staff during the process of injecting. Moreover, the administration is a like an operative procedure, which requires medical attendant and accompanying staff. Thus, there is need for an effective, pliable, easily administrable dosage form for the pet owner or any volunteer with required minimal attention.

Solving the problems of pets or animal's reluctance to swallow and poor compliance to known dosage forms has become the focus of the development of new dosage forms for oral administration of animals. Accordingly, it is also the main aim of the present inventors to provide a veterinary delivery system for effective absorption into the animal's vascular system through gastrointestinal tract, having dose delivery accuracy, easier way to administer and shows maximum therapeutic effect.

Keeping this in mind, the present inventors has developed oral films comprising effective amount of Antipruritic drugs and its pharmaceutically acceptable salts thereof and method of administering the same in treating, preventing or controlling allergic or atopic dermatitis in animals. The major advantages of this novel oral film dosage forms includes reduce difficulty or no difficulty while swallowing, avoid scratches and bites during administration, ease of administration, quick disintegration in the oral cavity besides they are less prone to being spit by the animals.

### SUMMARY OF THE INVENTION

In one embodiment, the present invention provides oral film of Antipruritic drugs and its pharmaceutically acceptable salts thereof for veterinary use.

In a preferred embodiment, the present invention provides composition of oral film of Antipruritic drugs and its pharmaceutically acceptable salts thereof for veterinary use, wherein the composition comprises at least one film forming polymer, plasticizer and one or more pharmaceutically acceptable excipients.

In another embodiment, the present invention provides process for preparing oral film of Antipruritic drugs and its pharmaceutically acceptable salts thereof for veterinary use, wherein the process may be selected from solvent casting, hot melt extrusion or printing technology.

In another embodiment, the present invention provides oral films of Antipruritic drugs and its pharmaceutically acceptable salts thereof used for the treatment, prevention or control of pruritus caused due to allergic or atopic dermatitis in animals.

### DETAILED DESCRIPTION OF THE INVENTION

References in the specification to “one embodiment,” “an embodiment,” “another embodiment,” “a preferred embodiment,” “one aspect,” “another aspect,” “preferred aspect” and the like, indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

As per United States Pharmacopoeia, Films are classified by the site of application. Accordingly, “Oral films” can be formulated to deliver medication to the mouth such as oral hygiene products or to deliver medication to the gastrointestinal tract for absorption. “Buccal films” and “sublingual films” are formulated to facilitate absorption through the proximal mucosal membranes avoiding first pass metabolism or degradation in the gastrointestinal tract and providing a quick onset of action.

“Oral film” according to the present invention may also referred as “Oral thin film,” “OTF,” “ODF,” “oral drug strip”, “oral strip”, “veterinary oral film”, “oral disintegrating film” or “oral dissolving film”.

The term “veterinary use” according to the present invention includes prevention or treatment of veterinary medical conditions in any animal other than human for which oral films of the present invention find useful. Animals herein unless otherwise specified does not limit to only domestic animals such as cats, dogs, guinea pigs, mice, horses, goats, rabbits, swine, apes, primates, hamsters, ferrets, reptiles, sheep, cows, and other non-human animals for which oral films of the present invention find useful.

The oral film, according to the present invention preferably disintegrates within about five minutes and more preferably within about 180 seconds upon contact with aqueous media or saliva in oral cavity.

An oral film according to the present invention is “non-mucoadhesive” in nature, means that the dosage form is not designed for administration of the active pharmaceutical agent through the oral mucosa i.e. the dosage form is not designed to adhere to the mucosal surfaces of the buccal cavity as an intact film or disintegrated film residue. In particular, the invention provides a non-mucoadhesive orally disintegrating film, able to disintegrate upon contact with aqueous media or saliva in oral cavity of the animal within about five minutes and more preferably within about 180 seconds.

The term “film” according to the present invention includes films, sheets and wafers, in any size and shape, including rectangular, square, or other desired shape. Preferably, they are distinct from pills, tablets, caplets or capsules and wherein the dosage from is like a thin strip of material. The films are generally sufficiently flexible to allow bending or even folding without breaking. The films described herein may be any desired thickness and size such that it may be placed into the oral cavity of the animal. The films may have a thickness of from about 20 microns to about 300 microns. Films may be in a single layer or they may be multi-layered, such as laminated or co-extruded films.

The term “disintegrating” according to present invention is defined as a state in which any residue of the oral film remaining on the screen of the test apparatus known in the art, or in the mouth of animal, is a soft mass having no palpably film core. The disintegration test does not imply complete solution of dosage unit or even of its active constituent, although a dissolved dosage unit would typically be completely disintegrated.

The term “dissolution” according to present invention is defined by the amount of active agent released from the oral film after oral administration or by in-vitro testing known in the art. An in-vitro dissolution test is to evaluate the performance of the product by measuring the amount of active agent dissolved in the dissolution medium. Standardized apparatus known in the art for in-vitro dissolution testing are: USP type I apparatus (basket), USP type II apparatus (Paddle), USP type V apparatus (Paddle over disk).

In one aspect the present invention is an oral film of Antipruritic drugs and its pharmaceutically acceptable salt thereof for veterinary use.

In another aspect the present invention provides composition and process for preparing oral film of Antipruritic drugs and its pharmaceutically acceptable salt thereof for veterinary use.

Antipruritic drugs according to the present invention are selected from the group comprising of antihistamines like Cetirizine, Diphenhydramine, Chlorphenamine, and Hydroxyzine; oral Glucocorticoids like Prednisolone, Prednisone, Methylprednisolone, Hydrocortisone, Betamethasone and Flumethasone; calcineurin inhibitor like Cyclosporine; Janus Kinase inhibitor (JAK-1) like Oclacitinib; fatty acids like Omega-3 and Omega-6; psychogenic drugs like Triflupromazine, Trimeprazine Tartrate or Alimemazine, Loratadine, Fluoxetine, Amitriptyline, Norepinephrine, Clemastine or Meclastin; opioid antagonists like Dextromethorphan and Naltrexone and any combinations thereof and its pharmaceutically acceptable salts thereof.

Pharmaceutically acceptable salt of Antipruritic drugs according to the present invention is selected from sodium, potassium, Magnesium, Fumarate, Sodium Phosphate, hydrochloride, hydrobromide, hydroiodide, nitrate, sulfate, bisulfate, Hemisulfate, phosphate, acid phosphate, succinate, acetate, lactate, citrate, acid citrate, tartrate, bitartrate, succinate, maleate, closylate, gluconate, saccharate, benzoate, methanesulfonate, ethanesulfonate, benzenesulfonate, p-toluenesulfonate, Mesylate, Besylate, Salicylate, disalicylate and pamoate.

Antipruritic drugs and its pharmaceutically acceptable salts thereof according to the present invention may be present in an amount of about 0.1% to about 75% by weight based on total weight of the composition.

In one preferred aspect, the present invention provides composition of oral film of Antipruritic drugs and its pharmaceutically acceptable salt thereof, wherein the composition comprises at least one film forming polymer, plasticizer and one or more pharmaceutically acceptable excipients.

According to the present invention, one or more pharmaceutically acceptable excipients are selected from the group comprising of suspending/thickening agents, fillers/bulking agents, disintegrating agents, stabilizers, surfactants, sweetening agents, taste masking agents, anti-foaming agents, flavoring agents and coloring agents and combinations thereof.

In another aspect, the present invention provides oral film of Antipruritic drugs and its pharmaceutically acceptable salts thereof comprises one or more Film forming polymers. The term “Polymers or Film forming polymers” according to the invention are responsible for imparting adequate mechanical properties to films, and they affect the film disintegration and/or dissolution in oral cavity. Film forming polymers used according to the present invention are not limited to hydrophilic or hydrophobic polymers.

Suitable non-limiting hydrophilic film forming polymers according to the present invention are selected from hydroxypropyl methylcellulose or Hypromellose, hydroxyethyl cellulose, hydroxypropyl cellulose, sodium carboxy methylcellulose, methyl cellulose, Hydroxypropyl ethylcellulose, povidone, copovidone, polydextrose, polyvinyl alcohol, polyvinyl acetate, polyethylene oxide, pullulan, sodium alginate, propylene glycol alginate, modified starch, gelatin, pectin, polyoxyethylene stearates, poly-epsilon caprolactone, polyglycolized glycerides, cyclodextrins, galactomannans, Polymerized rosin and combinations thereof.

Suitable non-limiting hydrophobic film forming polymers according to the present invention are selected from ethyl cellulose, Polymethacrylates, polyacrylic acid, methyl methacrylate copolymer, copolymers of acrylic acid and alkyl acrylate, carboxyvinyl copolymers, and combinations thereof.

Polymers used according to present invention are available in various viscosity ranges. Depending on the viscosity the quantity of the polymer is chosen.

Film forming polymer/s according to the present invention may be present in an amount of about 10% to about 75% by weight based on total weight of the composition.

In an aspect, the present invention provides oral film of Antipruritic drugs and its pharmaceutically acceptable salts thereof comprises plasticizers. The term “plasticizers” according to the present invention are responsible for mechanical properties of the film, such as tensile strength and decrease the fragility of film by decreasing the glass transition temperature of polymer. Suitable non-limiting plasticizers according to the present invention are selected from polyethylene glycol, propylene glycol, polyethylene-propylene glycol, glycerol/glycerin, glycerol monoacetate, glycerol diacetate, glycerol triacetate, triacetin, polysorbate, cetyl alcohol, sorbitol, sodium diethylsulfosuccinate, triethyl citrate, tributyl citrate, dimethyl phthalate, diethyl phthalate, dibutyl phthalate and combinations thereof.

Plasticizers according to the present invention may be present in an amount of about 1% to about 30% by weight based on total weight of the composition.

Suspending/thickening agents according to the present invention are responsible for improving the viscosity of the drug and excipient dispersion preparation and consistency of the oral film. Suitable non-limiting suspending/thickening agents according to the present invention are selected from maltodextrin, natural gums like xanthan gum, carrageen, locust bean gum, cellulose derivatives, dextrin, modified starch and combinations thereof.

Suspending/thickening agents according to the present invention may be present in an amount of about 1% to about 50% by weight based on total weight of the composition.

Suitable non-limiting filler/bulking agents according to the present invention are selected from Starches, anhydrous lactose, mannitol, Maltodextrin, dicalcium phosphate, calcium sulfate, cellulose, kaolin, sodium chloride, sorbitol, sucrose and combinations thereof.

Disintegrating agents according to the present invention are responsible for quick disintegration of oral film upon contact with aqueous media or saliva in oral cavity. Suitable non-limiting Disintegrating agents according to the present invention are selected from Starch, Colloidal silicon dioxide, microcrystalline cellulose, Crospovidone, Sodium Starch glycolate, Croscarmellose sodium and combinations thereof.

“Sweetening agent or Sweetener” according to the present invention enhances the palatable/taste and pleasurable factor, which makes the film relatively acceptable or agreeable to the patient. Suitable non-limiting sweeteners according to the present invention are selected from glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Ammonium Glycyrrhizinate, Stevia Rebaudiana (Stevioside); chloro derivatives of sucrose such as sucralose; sugar alcohols such as sorbitol, mannitol, maltodextrin, xylitol, and the like, hydrogenated starch hydrolysates and the synthetic sweetener 3,6-dihydro-6-methyl-1-1-1,2,3-oxathiazin-4-one-2,2-dioxide, particularly the potassium salt (Acesulfame-K), and sodium and calcium salts thereof, natural intensive sweeteners, protein based sweeteners such as thaumatoccous danielli (Thaumatin I and II) and combinations thereof.

“Taste masking agents” according to the present invention are responsible for blocking or diminishing the bitter taste of drug substance or excipients present in the film. Suitable non-limiting taste masking agents according to the present invention are selected from ion exchange resins, cyclodextrins and its derivatives, ready made available mixtures of taste mask enhancers and combinations thereof.

Suitable non-limiting buffering agents according to the present invention are selected from sodium carbonate, sodium bicarbonate, Citric Acid, Sodium Citrate, potassium carbonate, potassium bicarbonate, Ammonium Phosphate Dibasic, Sodium Phosphate Dibasic, sodium phosphate tribasic, potassium phosphate dibasic, potassium phosphate tribasic, Borate, calcium carbonate, magnesium carbonate, sodium hydroxide, magnesium hydroxide, potassium hydroxide, aluminium hydroxide, and combinations thereof.

Stabilizers according to the present invention are responsible for improving the shelf life by preventing the degradation of active agents. Suitable non-limiting stabilizers according to the present invention are selected from Tocopherol, Ascorbic acid, Citric acid, sodium bisulfite, sodium metabisulfite, propyl gallate, Potassium Metabisulfite, butylated hydroxytoluene and butylated hydroxyanisole and combinations thereof.

Suitable non-limiting surfactants according to the present invention are selected from cetyl alcohol, sodium lauryl sulfate, Polyoxyl 40 hydrogenated castor oil, Polyoxyl 15 hydroxystearate, Polysorbate 80, Macrogol stearate, Hydrogenated Castor Oil, Spans®, Tweens®, Ethoxylated oils, poloxamers and combinations thereof.

Suitable anti-foaming agents according to the present invention are selected from simethicone, dimethicone or any agent that removes air bubbles/entrapped air/void from film-forming compositions.

“Flavors” in the present invention are responsible for masking the bitter or nauseating taste of incorporated drug. Suitable non-limiting veterinary acceptable flavoring agents according to the present invention are selected from chicken, turkey, beef, pork, lamb, fish, egg, cheese, edible yeast, dairy products which are of either artificially available or naturally available or extracted from meat or organs of animals. Other non-limiting flavoring agents according to the present invention are selected from the group comprising of natural and synthetic flavoring liquids such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, pea nuts and citrus oils such as lemon, orange, grape, lime and grapefruit and fruit essences including apple, pear, peach, grape, strawberry, raspberry, cherry, plum, pineapple, apricot or other fruit flavors. Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin) and combinations thereof.

Suitable coloring agents according to the present invention are selected from the group comprising of food, drug and cosmetic colors (FD and C), drug and cosmetic colors (D and C), or external drug and cosmetic colors (Ext. D and C). These colors are dyes, their corresponding lakes, and certain natural and derived colorants. Other non-limiting examples of coloring agents according to present invention are selected from known azo dyes, organic or inorganic pigments, or coloring agents of natural origin and combinations thereof.

According to an aspect, some of the excipients used in the oral film of the present invention may have one or more functions i.e. an excipient used in oral film of the present invention may have multi-function like starch and modified starch used in the present invention may also act as a bulking agent and/or disintegrating agent; Maltodextrin used in the present invention may act as a sweetener and/or suspending/thickening agent and/or filler/bulking agent; Mannitol used in the present invention may act as a sweetener and/or as a filler/bulking agent; cellulose derivatives used in the present invention may act as a film forming polymer and/or suspending/thickening agent. Hence, a skilled person should not construe the limit of an excipient stated or illustrated in any aspect or embodiment or an example of oral film in the present invention to a single function.

In a preferred aspect, the present invention provides composition of oral film of Antipruritic drug(s) and its pharmaceutically acceptable salts thereof for veterinary use, wherein the composition comprises from about 0.1% to about 75% by weight of Antipruritic drug(s) and its pharmaceutically acceptable salts thereof.

In another preferred aspect, the present invention provides composition of oral film of Antipruritic drug(s) and its pharmaceutically acceptable salts thereof for veterinary use, wherein the composition comprises:
i. from about 0.1% to about 75% by weight of Antipruritic drug(s) and its pharmaceutically acceptable salts;
ii. from about 10% to about 75% by weight of at least one film forming polymer;
iii. from about 1% to about 30% by weight of plasticizer; and
iv. one or more pharmaceutically acceptable excipients;
   wherein the percentage (%) by weight is relative to the total weight of the composition.

In one aspect, the present invention provides process for preparing oral film of Antipruritic drugs and its pharmaceutically acceptable salts thereof for veterinary use, wherein the process may be selected from solvent casting, hot melt extrusion or printing technology.

Solvents used according to the present invention in the process for preparing oral film of Antipruritic drugs and its pharmaceutically acceptable salts thereof may be selected from purified water, a polar organic solvent including, but not limited to ethanol, dichloromethane, isopropanol, acetone, methylene chloride, or any combination thereof.

In one aspect, the present invention provides composition of oral film of Antipruritic drugs and its pharmaceutically acceptable salt thereof, wherein the composition comprises from about 0.1% to about 75% by weight of antipruritic drug(s) selected from antihistamines like Cetirizine, Diphenhydramine, Chlorphenamine, Hydroxyzine or combinations thereof and its pharmaceutically acceptable salts thereof for veterinary use.

In one aspect, the present invention provides composition of oral film of Antipruritic drugs and its pharmaceutically acceptable salt thereof, wherein the composition comprises from about 0.1% to about 75% by weight of antipruritic drug(s) selected from Glucocorticoids like Prednisolone, Prednisone, Methylprednisolone, Hydrocortisone, Betamethasone, Flumethasone or combinations thereof and its pharmaceutically acceptable salts thereof for veterinary use.

In one aspect, the present invention provides composition of oral film of Antipruritic drugs and its pharmaceutically acceptable salt thereof, wherein the composition comprises from about 0.1% to about 75% by weight of antipruritic drug(s) selected from psychogenic drugs like Triflupromazine, Trimeprazine or Alimemazine, Loratadine, Fluoxetine, Amitriptyline, Norepinephrine, Clemastine, meclastin or combinations thereof and its pharmaceutically acceptable salts thereof for veterinary use.

In one aspect, the present invention provides composition of oral film of Antipruritic drugs and its pharmaceutically acceptable salt thereof, wherein the composition comprises from about 0.1% to about 75% by weight of antipruritic drug(s) selected from opioid antagonists like Dextromethorphan, Naltrexone or combinations thereof and its pharmaceutically acceptable salts thereof for veterinary use.

In an aspect, the present invention provides oral film of Oclacitinib and its pharmaceutically acceptable salt thereof.

In an aspect, the present invention provides composition of oral film of Oclacitinib and its pharmaceutically acceptable salt thereof, wherein the composition comprises at least one film forming polymer, plasticizer and one or more pharmaceutically acceptable excipients.

In an aspect, the present invention provides process for preparing oral film of Oclacitinib and its pharmaceutically acceptable salt thereof, wherein the process may be selected from solvent casting, hot melt extrusion or printing technology.

In a preferred aspect, the present invention provides composition of oral film of Oclacitinib and its pharmaceutically acceptable salt thereof, wherein the composition comprises from about 0.1% to about 75% by weight of Oclacitinib and its pharmaceutically acceptable salt.

In another preferred aspect, the present invention provides composition of oral film of Oclacitinib and its pharmaceutically acceptable salt thereof, wherein the composition comprises:
i. from about 0.1% to about 75% by weight of Oclacitinib and its pharmaceutically acceptable salt;
ii. from about 10% to about 75% by weight of at least one film forming polymer;
iii. from about 1% to about 30% by weight of plasticizer; and
iv. one or more pharmaceutically acceptable excipients;
   wherein the percentage (%) by weight is relative to the total weight of the composition.

In an aspect, the present invention provides oral film of Cyclosporine and its pharmaceutically acceptable salt thereof.

In an another preferred aspect, the present invention provides composition of oral film of Cyclosporine and its pharmaceutically acceptable salt thereof, wherein the composition comprises at least one film forming polymer, plasticizer and one or more pharmaceutically acceptable excipients.

In a further aspect, the present invention provides process for preparing oral film of Cyclosporine and its pharmaceutically acceptable salt thereof, wherein the process may be selected from solvent casting, hot melt extrusion or printing technology.

In a preferred aspect, the present invention provides composition of oral film of Cyclosporine and its pharmaceutically acceptable salt thereof, wherein the composition comprises from about 0.1% to about 75% by weight of Cyclosporine and its pharmaceutically acceptable salt.

In another preferred aspect, the present invention provides composition of oral film of Cyclosporine and its pharmaceutically acceptable salt thereof, wherein the composition comprises:
i. from about 0.1% to about 75% by weight of Cyclosporine and its pharmaceutically acceptable salt;
ii. from about 10% to about 75% by weight of at least one film forming polymer;
iii. from about 1% to about 30% by weight of plasticizer; and
iv. one or more pharmaceutically acceptable excipients;
   wherein the percentage (%) by weight is relative to the total weight of the composition.

In an aspect, the present invention provides oral film of Cetirizine and its pharmaceutically acceptable salts thereof.

In an another preferred aspect, the present invention provides composition of oral film of Cetirizine and its pharmaceutically acceptable salts thereof, wherein the composition comprises at least one film forming polymer, plasticizer and one or more pharmaceutically acceptable excipients.

In a further aspect, the present invention provides process for preparing oral film of Cetirizine and its pharmaceutically acceptable salt thereof, wherein the process may be selected from solvent casting, hot melt extrusion or printing technology.

In a preferred aspect, the present invention provides composition of oral film of Cetirizine and its pharmaceutically acceptable salt thereof, wherein the composition comprises from about 0.1% to about 75% by weight of Cetirizine and its pharmaceutically acceptable salt.

In another preferred aspect, the present invention provides composition of oral film of Cetirizine and its pharmaceutically acceptable salt thereof, wherein the composition comprises:
i. from about 0.1% to about 75% by weight of Cetirizine and its pharmaceutically acceptable salt;
ii. from about 10% to about 75% by weight of at least one film forming polymer;
iii. from about 1% to about 30% by weight of plasticizer; and
iv. one or more pharmaceutically acceptable excipients;
   wherein the percentage (%) by weight is relative to the total weight of the composition.

In an aspect, the present invention provides oral film of Prednisone and its pharmaceutically acceptable salt thereof.

In an another preferred aspect, the present invention provides composition of oral film of Prednisone and its pharmaceutically acceptable salt thereof, wherein the composition comprises at least one film forming polymer, plasticizer and one or more pharmaceutically acceptable excipients.

In a further aspect, the present invention provides process for preparing oral film of Prednisone and its pharmaceutically acceptable salt thereof, wherein the process may be selected from solvent casting, hot melt extrusion or printing technology.

In a preferred aspect, the present invention provides composition of oral film of Prednisone and its pharmaceutically acceptable salt thereof, wherein the composition comprises from about 0.1% to about 75% by weight of Prednisone and its pharmaceutically acceptable salt.

In another preferred aspect, the present invention provides composition of oral film of Prednisone and its pharmaceutically acceptable salt thereof, wherein the composition comprises:
i. from about 0.1% to about 75% by weight of Prednisone and its pharmaceutically acceptable salt;
ii. from about 10% to about 75% by weight of at least one film forming polymer;
iii. from about 1% to about 30% by weight of plasticizer; and
iv. one or more pharmaceutically acceptable excipients;
   wherein the percentage (%) by weight is relative to the total weight of the composition.

In an aspect, the present invention provides oral film of Fluoxetine and its pharmaceutically acceptable salt thereof.

In an another preferred aspect, the present invention provides composition of oral film of Fluoxetine and its pharmaceutically acceptable salt thereof, wherein the composition comprises at least one film forming polymer, plasticizer and one or more pharmaceutically acceptable excipients.

In a further aspect, the present invention provides process for preparing oral film of Fluoxetine and its pharmaceutically acceptable salt thereof, wherein the process may be selected from solvent casting, hot melt extrusion or printing technology.

In a preferred aspect, the present invention provides composition of oral film of Fluoxetine and its pharmaceutically acceptable salt thereof, wherein the composition comprises from about 0.1% to about 75% by weight of Fluoxetine and its pharmaceutically acceptable salt.

In another preferred aspect, the present invention provides composition of oral film of Fluoxetine and its pharmaceutically acceptable salt thereof, wherein the composition comprises:
i. from about 0.1% to about 75% by weight of Fluoxetine and its pharmaceutically acceptable salt;
ii. from about 10% to about 75% by weight of at least one film forming polymer;
iii. from about 1% to about 30% by weight of plasticizer; and
iv. one or more pharmaceutically acceptable excipients;
wherein the percentage (%) by weight is relative to the total weight of the composition.

In an aspect, the present invention provides oral films of Antipruritic drugs and its pharmaceutically acceptable salts thereof for the treatment, prevention or control of pruritus caused due to allergic or atopic dermatitis in animals.

In one preferred aspect, the present invention provides oral films of Oclacitinib and its pharmaceutically acceptable salt thereof for the treatment, prevention or control of pruritus caused due to allergic or atopic dermatitis in animals.

In another preferred aspect, the present invention provides oral films of Cyclosporine and its pharmaceutically acceptable salt thereof for the treatment, prevention or control of pruritus caused due to allergic or atopic dermatitis in animals.

In another preferred aspect, the present invention provides oral films of Cetirizine and its pharmaceutically acceptable salt thereof for the treatment, prevention or control of pruritus caused due to allergic or atopic dermatitis in animals.

In another preferred aspect, the present invention provides oral films of Prednisone and its pharmaceutically acceptable salt thereof for the treatment, prevention or control of pruritus caused due to allergic or atopic dermatitis in animals.

In another preferred aspect, the present invention provides oral films of Fluoxetine and its pharmaceutically acceptable salt thereof for the treatment, prevention or control of pruritus caused due to allergic or atopic dermatitis in animals.

The said invention is further illustrated by following non-limiting examples: which are set forth to aid in an understanding of the invention, and are not intended, and should not be construed, to limit in any way the invention set forth in the claims that follow thereafter. A person skilled in the art will readily recognize the various modifications and variations that may be performed without altering the scope of the present invention. Such modifications and variations are encompassed within the scope of the invention and the examples do not in any way limit the scope of the invention.

### Example 1

### Manufacturing Formula:

| | | Quantity per unit (mg) | | | |
|---|---|---|---|---|---|
| S. No. | Ingredients | 1A | 1B | 1C | 1D |
| 1 | Oclacitinib | 16 | 16 | 16 | 16 |
| 2 | Hydroxypropyl | 30-80 | — | 30-80 | — |
| | methylcellulose | | | | |
| 3 | Hydroxypropyl cellulose | — | 30-80 | — | 30-80 |
| 4 | Microcrystalline Cellulose | 5-15 | 5-15 | 5-15 | 5-15 |
| 5 | Polyethylene glycol | 4-8 | 4-8 | — | — |
| 6 | Polysorbate 80 | — | — | 4-8 | 4-8 |
| 7 | Propylene glycol | 4-8 | 4-8 | 4-8 | 4-8 |
| 8 | Flavour | 4-6 | 4-6 | 4-6 | 4- 6 |
| 9 | Colorant | 0.05- | 0.05- | 0.05- | 0.05- |
| | | 0.10 | 0.10 | 0.10 | 0.10 |
| 10 | Purified water | q.s | q.s | q.s | q.s |
| 11 | Ethanol | q.s | q.s | q.s | q.s |
| Film Weight | | 63.05- | 63.05- | 63.05- | 63.05- |
| | | 133.1 | 133.1 | 133.1 | 133.1 |

### Brief Manufacturing Procedure:

1. Dispense all the ingredients.

2. Mix purified water and ethanol in a suitable stainless steel vessel.

3. Add Hydroxypropyl methylcellulose or Hydroxypropyl cellulose to step 2 under stirring and continue stirring till a homogenous dispersion is obtained.

4. Optionally add Polysorbate 80 and/or Polyethylene glycol to dispersion of step 3 under stirring and continue stirring till a homogenous dispersion is obtained.

5. Add Oclacitinib to the dispersion of step 4 under stirring and continue stirring till a homogenous dispersion is obtained.

6. Add Microcrystalline Cellulose to dispersion of step 5 under stirring and continue stirring till a homogenous dispersion is obtained.

7. Add Propylene glycol to dispersion of step 6 under stirring and continue stirring till a homogenous dispersion is obtained.

8. Add colorant to dispersion of step 7 under stirring and continue stirring till a homogenous dispersion is obtained.

9. Add, flavor to dispersion of step 8 under stirring and continue stirring till a homogenous dispersion is obtained.

10. Homogenize the dispersion of step 9 using a homogenizer to obtain a homogenous dispersion.

11. If any foam observed, de-aerate the dispersion by applying vacuum under slow stirring.

12. The dispersion of step 10 or 11 is casted using a film casting machine where the dispersion is spreaded on a backing film to form a thin wet film which passed through a drying zone.

13. The dried film is cut into a desired size by using slitter.

14. Pack the film into suitable pouches/sachets

### Example 2

### Manufacturing Formula:

| | | Quantity per unit (mg) | | | |
|---|---|---|---|---|---|
| S. No. | Ingredients | 2A | 2B | 2C | 2D |
| 1 | Cyclosporine | 25 | 25 | 25 | 25 |
| 2 | Polyvinyl alcohol | 30-60 | 30-60 | 30-60 | 30-80 |
| 3 | Modified starch | 8-16 | 8-16 | 8-16 | 8-16 |
| 4 | Colloidal silicon | 5-15 | 5-15 | 5-15 | 5-15 |
| | dioxide | | | | |
| 5 | Polyethylene glycol | 4-8 | — | 4-8 | 4-8 |
| 6 | Polysorbate 80 | — | 4-8 | 4-8 | 4-8 |
| 7 | Propylene glycol | 4-8 | 4-8 | 4-8 | 4-8 |
| 8 | Flavour | 4-6 | 4-6 | 2-4 | 2-4 |
| 9 | Colorant | 0.05- | 0.05- | 0.05- | 0.05- |
| | | 0.10 | 0.10 | 0.10 | 0.10 |
| 10 | Ethanol | q.s | q.s | q.s | q.s |
| Film Weight | | 80.05- | 80.05- | 82.05- | 82.05- |
| | | 138.1 | 138.1 | 144.1 | 144.1 |

### Brief Manufacturing Procedure:

1. Dispense all the ingredients.

2. Add ethanol in a suitable stainless steel vessel.

3. Add polyvinyl alcohol to step 2 under stirring and continue stirring till a homogenous dispersion is obtained.

4. Add modified starch to step 3 under stirring and continue stirring till a homogenous dispersion is obtained.

5. Add Polysorbate 80 and/or Polyethylene glycol to dispersion of step 4 under stirring and continue stirring till a homogenous dispersion is obtained.

6. Add Cyclosporine under stirring to step 5 and continue stirring till a homogenous dispersion is obtained.

7. Add Colloidal silicon dioxide to dispersion of step 6 under stirring and continue stirring till a homogenous dispersion is obtained.

8. Add Propylene glycol to dispersion of step 7 under stirring and continue stirring till a homogenous dispersion is obtained.

9. Add colorant to dispersion of step 8 under stirring and continue stirring till a homogenous dispersion is obtained.

10. Add flavor to dispersion of step 9 under stirring and continue stirring till a homogenous dispersion is obtained.

11. Homogenize the dispersion of step 10 using a homogenizer to obtain a homogenous dispersion.

12. If any foam observed, de-aerate the dispersion of step 11 by applying vacuum under slow stirring.

13. The dispersion of step 11 or 12 is casted using a film casting machine where the dispersion is spreaded on a backing film to form a thin wet film which passed through a drying zone.

14. The dried film is cut into a desired size by using slitter.

15. Pack the film into suitable pouches/sachets.

### Example 3

| S. No | Ingredient Name | % w/w | mg/unit |
|---|---|---|---|
| 1 | Oclacitinib | 17.78 | 16.00 |
| 2 | Hypromellose E15 | 55.56 | 50.00 |
| 3 | Microcrystalline cellulose | 11.11 | 10.00 |
| 4 | Polysorbate-80 | 4.44 | 4.00 |
| 5 | Propylene glycol | 6.67 | 6.00 |
| 6 | Chicken flavour | 4.44 | 4.00 |
| 7 | Ethanol | q.s | q.s |
| 8 | Purified water | q.s | q.s |

| | | |
|---|---|---|
| Film weight (mg) | 100.00 | 90.00 |

### Brief Manufacturing Procedure:

1. Ethanol was taken in a suitable stainless steel vessel.

2. Added Oclacitinib to the step 1 and stirred with a suitable stirrer to get a homogenous dispersion.

3. Taken required quantity of purified water in stainless steel vessel and added Hypromellose E15 under stirring and continued stirring till a homogenous dispersion is obtained.

4. Added microcrystalline cellulose and flavor to dispersion of step 3 under stirring and continued stirring till a homogenous dispersion is obtained.

5. Added Polysorbate 80 to dispersion of step 4 under stirring and continued stirring till a homogenous dispersion is obtained.

6. Added Propylene glycol to step 5 under stirring and continued stirring till a homogenous dispersion is obtained.

7. Added step 6 to step 2 under stirring and continued stirring till a homogenous dispersion is obtained.

8. The suspension was homogenized using a homogenizer to obtain a homogenous suspension.

9. De-aerated the dispersion by applying vacuum under slow stirring.

10. The dispersion is casted using a film casting machine where the dispersion is spreaded on a backing film to form a thin wet film which passed through a drying zone.

11. The dried film is cut into a desired size by using slitter.

12. Pack the film into suitable pouches/sachets

Disintegration study: The obtained oral film of Example 3 is tested for disintegration time using static method, wherein the oral film is placed in a petri dish containing water in a static condition and the time taken for disintegration is observed.
Time taken for disintegration—60 seconds

Dissolution study: Also the obtained film of Example 3 is tested for dissolution in USP type 5 (Paddle over Disc) apparatus, 900 ml volume, 75 rpm using 0.1N HCl as dissolution medium and the results are give below:

| Time (minutes) | % Cumulative drug dissolved |
|---|---|
| 5 | 97 |
| 10 | 97 |
| 15 | 98 |

Form the above results, the oral film of present invention has shown more than 95% of drug is released within 10 minutes, which shows faster release of drug.

Therefore, oral films of the present invention formulated according to an embodiment has shown quick disintegration and dissolution of drug, which solves the problems mentioned above like difficulty of administration, swallowing, dosing errors etc. in animals.

1. An oral film of Antipruritic drug(s) and its pharmaceutically acceptable salt thereof for veterinary use.

2. The oral film as claimed in claim 1, wherein the Antipruritic drug(s) is selected from Oclacitinib, Cyclosporine, Cetirizine, Diphenhydramine, Chlorphenamine, Hydroxyzine, Prednisolone, Prednisone, Methylprednisolone, Hydrocortisone, Betamethasone, Flumethasone, Omega-3 fatty acid, Omega-6 fatty acid, Triflupromazine, Trimeprazine, Alimemazine, Loratadine, Fluoxetine, Amitriptyline, Norepinephrine, Clemastine, Meclastin, Dextromethorphan, Naltrexone and combinations thereof and its pharmaceutically acceptable salt thereof.

3. The oral film as claimed in claim 1, wherein the film comprises:
a. Antipruritic drug(s) and its pharmaceutically acceptable salt thereof selected from Oclacitinib, Cyclosporine, Cetirizine, Diphenhydramine, Chlorphenamine, Hydroxyzine, Prednisolone, Prednisone, Methylprednisolone, Hydrocortisone, Betamethasone, Flumethasone, Omega-3 fatty acid, Omega-6 fatty acid, Triflupromazine, Trimeprazine, Alimemazine, Loratadine, Fluoxetine, Amitriptyline, Norepinephrine, Clemastine, Meclastin, Dextromethorphan, Naltrexone and combinations thereof;
b. at least one film forming polymer;
c. plasticizer; and
d. one or more pharmaceutically acceptable excipients.

4. The oral film as claimed in claim 3, wherein the film forming polymer is selected from hydroxypropyl methylcellulose, hydroxyethyl cellulose, hydroxypropyl cellulose, sodium carboxy methylcellulose, methyl cellulose, Hydroxypropyl ethylcellulose, povidone, copovidone, polydextrose, polyvinyl alcohol, polyvinyl acetate, polyethylene oxide, pullulan, sodium alginate, propylene glycol alginate, modified starch, gelatin, pectin, polyoxyethylene stearates, poly-epsilon caprolactone, polyglycolized glycerides, cyclodextrins, galactomannans, polymerized rosin, ethyl cellulose, Polymethacrylates, polyacrylic acid, methyl methacrylate copolymer, copolymers of acrylic acid and alkyl acrylate, carboxyvinyl copolymers and combinations thereof.

5. The oral film as claimed in claim 3, wherein the plasticizer is selected from polyethylene glycol, propylene glycol, polyethylene-propylene glycol, glycerol/glycerin, glycerol monoacetate, glycerol diacetate, glycerol triacetate, triacetin, polysorbate, cetyl alcohol, sorbitol, sodium diethylsulfosuccinate, triethyl citrate, tributyl citrate, dimethyl phthalate, diethyl phthalate, dibutyl phthalate and combinations thereof.

6. The oral film as claimed in claim 3, wherein one or more pharmaceutically acceptable excipients are selected from suspending/thickening agents, disintegrating agents, fillers/bulking agents, stabilizers, surfactants, sweetening agents, taste masking agents, buffering agents, anti-foaming agents, flavoring agents, and coloring agents.

7. The oral film as claimed in claim 1, wherein the process of preparing oral film of Antipruritic drug(s) and its pharmaceutically acceptable salt thereof is by solvent casting, hot melt extrusion or printing technology.

8. The oral film as claimed in claim 1, wherein the oral film of Antipruritic drug(s) and its pharmaceutically acceptable salt thereof is administered to any non-human animal for the treatment, prevention or control of pruritus caused due to allergic or atopic dermatitis.

9. The oral film as claimed in claim 3, wherein the process of preparing oral film of Antipruritic drug(s) and its pharmaceutically acceptable salt thereof is by solvent casting, hot melt extrusion or printing technology.

10. The oral film as claimed in claim 3, wherein the oral film of Antipruritic drug(s) and its pharmaceutically acceptable salt thereof is administered to any non-human animal for the treatment, prevention or control of pruritus caused due to allergic or atopic dermatitis.
